(19) United States
(12) Patent Application Publication
(10) Pub. No.: US 2013/0102682 A1
(43) Pub. Date: Apr. 25, 2013

(54) FINGOLIMOD IN THE FORM OF A SOLID SOLUTION

(71) Applicants: Jana Paetz, Bonn (DE); Dominique Meergans, Munich (DE)

(72) Inventors: Jana Paetz, Bonn (DE); Dominique Meergans, Munich (DE)

(73) Assignee: Ratiopharm GMBH, Ulm (DE)

(21) Appl. No.: 13/642,166

(22) PCT Filed: Apr. 21, 2011
(86) PCT No.: PCT/EP11/02052

§ 371 Date: Dec. 20, 2012

(30) Foreign Application Priority Data
Apr. 22, 2010 (DE) 10 2010 017 945.0

(51) Int. Cl. A61K 31/137; A61J 3/10

(52) U.S. Cl. CPC A61K 31/137; A61J 3/10
USPC 514/653

(57) ABSTRACT

The invention relates to an intermediate containing fingolimod and matrix material, wherein the fingolimod is present in the matrix material in the form of a solid solution. The invention also relates to granules and pharmaceutical formulations containing fingolimod in the form of a solid solution in matrix material. The subject matter of the invention further relates to methods of preparing a solid solution of fingolimod or of an intermediate, and also granules and pharmaceutical formulations containing fingolimod in the form of a solid solution.

The invention relates to an intermediate containing fingolimod and matrix material, wherein the fingolimod is present in the matrix material in the form of a solid solution. The invention also relates to granules and pharmaceutical formulations containing fingolimod in the form of a solid solution in matrix material. The subject matter of the invention further comprises methods of preparing a solid solution of fingolimod or of an intermediate, and also granules and pharmaceutical formulations containing fingolimod in the form of a solid solution.

Fingolimod, which is also referred to as “FTY720”, is a synthetic imitation of myriocin, a metabolic product of the fungus Isaria sinclairii. Fingolimod is a modulator of the sphingosine-1 phosphate receptor, which, after phosphorylation, can bind sphingosine-1 phosphate receptors, especially of T and B-lymphocytes. This inhibits the migration of lymphocytes from the lymph nodes into the blood and hence reduces their distribution in the central nervous system. Inflammatory T-lymphocytes are possible triggers for the destruction of the neural myelin sheaths, which are responsible for the typical symptoms of multiple sclerosis. For this reason, fingolimod is a possible means for the treatment of multiple sclerosis and especially for the treatment of patients with relapsing-remitting multiple sclerosis.

The IUPAC name of fingolimod is 2-amino-2-(2-[4-octylphenyl]ethyl)-1,3-propane diol. The chemical structure of fingolimod is shown in formula (I) below:

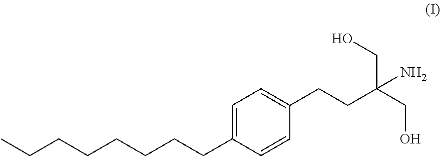 [SMILES: CCCCCCCCc1ccc(CCC(N)(CO)CO)cc1]

The synthesis of fingolimod is described in, for example, the European patent application EP 0 627 406.

Fingolimod is currently undergoing Phase III clinical trials, in which doses of 0.5 and 1.25 mg are being administered orally once a day. For the treatment of multiple sclerosis, doses ranging from 0.25 to 2.5 mg, i.e. very small amounts, are generally contemplated.

The proportion of the active agent in the total weight of the formulation (incl. active agent), or the formulation unit, especially in the case of formulations for oral administration, is typically in the range of only a few percent by weight, such as 0.25 to 4% by weight. This small proportion of active agent can lead to considerable problems with regard to the uniformity of the content of active agent in the individual formulation units. Different contents of active agent can lead to undesirable side-effects and changes in the bio-availability and efficacy. This problem is further aggravated by the fact that fingolimod exhibits relatively poor flowability and only forms homogeneous mixtures with standard pharmaceutical excipients to an inadequate extent.

The Ph. Eur. 6.0 section 2.9.6 therefore prescribes a uniformity test for the content of active agent in formulation units. According to that test, each individual content of 10 units must lie between 85 and 115 percent of the average content. If more than one individual content lies outside that limit or if one individual content lies outside the limits of 75 to 125 percent of the average content, the formulation units do not pass the test.

One problem of the present invention was therefore to provide the active agent in a form possessing good flowability and thus making it possible for it to be processed not only into capsules, but also to ensure good compression into tablets. It is also the intention to provide the active agent in a form which does not have a tendency to agglomerate. In addition, it is intended to enable an even distribution of the active agent. Furthermore, the intention is to provide fingolimod in a form that makes it possible to achieve a high level of uniformity of content (content uniformity), in pharmaceutical formulations and especially with a low content of active agent (drug load).

It has unexpectedly been possible to solve the problems by converting fingolimod into a solid solution.

The subject matter of the invention is therefore an intermediate containing fingolimod and matrix material, the fingolimod being present in the form of a solid solution in the matrix material. The weight ratio of fingolimod to matrix material is preferably 1:1 to 1:200. The intermediate is a solid solution of fingolimod in stabilised form.

The subject matter of the invention further relates to various methods of preparing a solid solution of fingolimod in the form of the intermediate of the invention, and a method of preparing granules from the intermediate and a method of preparing a pharmaceutical formulation from the intermediate and/or granules.

Finally, the subject matter of the invention also comprises granules and pharmaceutical formulations containing the fingolimod of the invention in the form of a solid solution or in the form of the intermediate of the invention.

Furthermore, the subject matter of the invention also comprises pharmaceutical formulations containing the fingolimod of the invention in the form of a solid solution or in the form of the intermediate of the invention for the treatment of multiple sclerosis, preferably relapsing-remitting multiple sclerosis. In addition, one subject matter of the invention is the pharmaceutical formulation of the invention for administration with a pharmaceutical formulation containing an active agent different from fingolimod.

It has transpired that the provision of fingolimod in the form of a solid solution makes it advantageously possible to prepare pharmaceutical formulations with different, very small contents of active agent in such a way that they exhibit very good uniformity of the content of active agent.

Furthermore, it has transpired that thanks to their good flowability, bulk density and compressibility, the intermediates of the invention are very advantageous in their use for preparing pharmaceutical formulations.

In addition, it has surprisingly been found that by using pharmaceutical formulations containing the intermediates of the invention, dependencies of the absorption of the active agent on the intake of food (“food effect”) can be eliminated or at least reduced substantially. The intermediates of the invention and the pharmaceutical formulations containing them can release the active agent independently of the pH.

Another particular advantage of these intermediates and the formulations containing them is that they can advantageously be administered with other medicaments, i.e. pharmaceutical formulations with an active agent different from fingolimod, without the absorption of fingolimod being impaired. This applies especially to medicaments which are suitable for influencing the pH at the site where active agent is absorbed.

In the context of the present invention, the term “fingolimod” comprises 2-amino-2-(2-[4-octylphenyl]ethyl)-1,3-propane diol according to the above formula (I). In addition, the term “fingolimod” comprises all the pharmaceutically acceptable salts, hydrates and/or solvates thereof. Acid addition salts are the salts preferably used. Examples of suitable salts are hydrochlorides, carbonates, hydrogen carbonates, acetates, lactates, butyrates, propionates, sulphates, methane sulphonates, citrates, tartrates, nitrates, sulphonates, oxalates and/or succinates. Fingolimod hydrochloride is particularly preferably used.

The term “solid solution” is to be understood in the context of this invention as meaning that fingolimod is distributed in a molecularly disperse manner in a matrix which is present in a solid state at 25° C. and a pressure of 101 kPa.

It is preferable that the intermediate of the invention (containing fingolimod in the form of a solid solution) should contain less than 15% by weight, more preferably less than 5% by weight, of crystalline fingolimod with a crystal or crystallite size of more than 300 nm, based on the total weight of the fingolimod present in the intermediate. It is further preferred that the intermediate of the invention (containing fingolimod in the form of a solid solution) contains substantially no crystalline fingolimod. In particular, the intermediate of the invention contains less than 15% by weight, more preferably less than 5% by weight, of crystalline fingolimod of any crystal or crystallite size, based on the total weight of the fingolimod present in the intermediate. The crystalline proportion is determined by means of quantitative X-ray diffractometry according to the method of Hermans and Weidinger.

“Crystalline” generally means substances the smallest components of which build up crystal structures, but also substances consisting of tiny crystallites. The atoms, ions or molecules which the respective crystal substance consists of form characteristic arrangements which are repeated periodically, so that they exhibit a long-range order. Crystals are thus anisotropic. Crystalline substances can be identified experimentally by means of X-ray diffraction, which reveals clearly defined interference patterns for crystalline substances. In contrast to this, X-ray diffraction performed on amorphous substances does not reveal clearly defined interferences for them, but normally only a few diffuse interferences with small diffraction angles.

It is therefore preferable for “molecularly disperse” to be understood as meaning that X-ray diffraction analysis of the fingolimod contained in the embodiments of the invention does not reveal any clearly defined interference patterns, but at most only a few diffuse interferences with small diffraction angles.

It is also preferable for “molecularly disperse” to be understood as meaning that the intermediate of the invention contains substantially no, preferably less than 15, 10, 5 or 2% by weight, fingolimod particles with a particle size of more than 1 μm, preferably less than 15, 10, 5 or 2% by weight of fingolimod particles with a particle size of more than 800 nm, preferably less than 15, 10, 5 or 2% by weight of fingolimod particles with a particle size of more than 500 nm, preferably less than 15, 10, 5 or 2% by weight of fingolimod particles with a particle size of more than 300 nm, more preferably less than 15, 10, 5 or 2% by weight of fingolimod particles with a particle size of more than 200 nm, and most preferably less than 15, 10, 5 or 2% by weight of fingolimod particles with a particle size of more than 100 nm.

The particle size is determined in this context by means of confocal Raman spectroscopy. The measuring system preferably consists of an NTEGRA-Spektra Nanofinder ex NT-MDT.

In a preferred embodiment, the intermediate of the invention is consequently a “single-phase” intermediate. As a monophase system, the intermediate is defined by reference to a common glass transition point of the excipient and the active agent. This can be analysed by means of DSC.

In the context of this invention, the solid solution of fingolimod of the invention is present in stabilised form, namely in the form of an intermediate, containing molecularly disperse fingolimod and a matrix material. In particular, the intermediate of the invention consists substantially of molecularly disperse fingolimod and matrix material. If—as described below—a crystallisation inhibitor is used in addition, the intermediate of the invention may consist substantially of molecularly disperse fingolimod, matrix material and crystallisation inhibitor. The expression “substantially” in this case indicates that small amounts of solvent etc. may also be present where applicable.

The matrix material is generally a substance which is suitable for stabilising fingolimod in the form of a solid solution. The matrix material is preferably a polymer. In addition, the matrix material also includes substances which behave like polymers. Furthermore, the matrix material also includes solid, non-polymeric compounds which preferably contain polar side groups. Finally, the term “matrix material” also includes surfactants, especially surfactants which are present in solid form at room temperature. The matrix material preferably has a melting point of 50° C. or more. If the matrix material is a mixture of substances, it is preferable that each substance in the mixture should have a melting point of 50° C. or more.

A further subject matter of the invention is a method of identifying a pharmaceutical excipient which is suitable as a matrix material for a solid fingolimod solution and which can hence be used for preparing the intermediate of the invention. The method comprises the steps of:
a) preparing fingolimod, a pharmaceutical excipient which is present in a solid aggregate state at 25° C., and a 1:1 mixture of fingolimod and excipient;
b) twice heating up the solid excipient by means of DSC and identifying the glass transition temperature of the excipient (Tg_{Excip});
c) twice heating up the active agent fingolimod by means of DSC and identifying the glass transition temperature of the active agent (Tg_{Fingo});
d) twice heating up a 1:1 mixture of fingolimod and excipient by means of DSC and identifying the glass transition temperature of the mixture (Tg_{Mix}), and
e) selecting the excipient as “suitable” provided that Tg_{Mix} is between Tg_{Excip} and Tg_{Fingo}.

In this case, two heating curves are recorded by means of DSC (Differential Scanning calorimetry, Dynamic Differential calorimetry). The curves are usually recorded from 20° C. to no more than 20° C. below the decomposition range of the substance to be tested. The term “1:1-mixture” refers to a mixture of 50% by weight fingolimod and 50% by weight excipient, which is prepared by mixing.

For this purpose a Mettler Toledo DSC 1 apparatus can be used. The work is performed at a heating rate of 1-20° C./min, preferably 10° C./min, and at a cooling rate of 5-25° C./min, preferably 15° C./min.

The subject matter of the invention is also an intermediate of molecularly disperse fingolimod and a pharmaceutical excipient as the matrix material, the excipient being identified in accordance with the above method, and wherein the weight ratio of fingolimod to matrix material is preferably 1:1 to 1:200.

The matrix material used for the preparation of the intermediate of the invention is preferably a polymer, or the matrix material comprises a polymer.

The excipient that can be used for the preparation of the intermediate, or the polymer that can be used for the preparation of the intermediate, preferably has a melting point (Ts) or a glass transition temperature (Tg) of more than 20° C., preferably 20° C. to 220° C., more preferably 40° C. to 180° C., more preferably 40° C. to 100° C. By immobilisation, a polymer with a Tg selected accordingly is particularly advantageous in preventing the reformation of the molecular fingolimod dispersion into colloids or particles.

The term “glass transition temperature” (Tg) is used to describe the temperature at which amorphous or partially crystalline excipients or polymers change from the solid state to the liquid state. In the process, a distinct change in physical parameters, e.g. hardness and elasticity, occurs. Below the Tg, an excipient or polymer is usually glassy and hard, whereas above the Tg, it changes into a rubber-like to viscous state. The glass transition temperature is determined in the context of this invention by means of dynamic differential scanning calorimetry (DSC).

For this purpose a Mettler Toledo DSC 1 apparatus can be used. The work is performed at a heating rate of 1-20° C./min, preferably 10° C./min, and at a cooling rate of 5-25° C./min, preferably 15° C./min.

In addition, the polymer which can be used for the preparation of the intermediate preferably has a number-average molecular weight of 1,000 to 250,000 g/mol, more preferably from 2,000 to 100,000 g/mol, and particularly preferably 4,000 to 50,000 g/mol. When the polymer used in the preparation of the intermediate is dissolved in (distilled) water in an amount of 2% by weight, the resulting solution preferably has a viscosity of 0.1 to 18 mPaxs, more preferably 0.5 to 15 mPaxs, especially 2 to 8 mPaxs, measured at 25° C. The viscosity is measured here in accordance with the European Pharmacopoeia (Ph. Eur.), 6th edition, section 2.2.10.

Hydrophilic polymers are preferably used for the preparation of the intermediate. This refers to polymers which possess hydrophilic groups. Examples of suitable hydrophilic groups are hydroxy, alkoxy, acrylate, methacrylate, sulphonate, carboxylate and quaternary ammonium groups. Hydroxy groups are preferable.

The intermediate of the invention may, for example, comprise the following hydrophilic polymers as matrix material: polyvinyl pyrrolidone, polyvinyl acetate (PVAC), polyvinyl alcohol (PVA), polymers of acrylic acid and their salts, polyacrylamide, polymethacrylates, vinyl pyrrolidone/vinyl acetate copolymers (such as Kollidon® VA64, BASF), polyalkylene glycols, such as polypropylene glycol or preferably polyethylene glycol, co-block polymers of polyethylene glycol, especially co-block polymers of polyethylene glycol and polypropylene glycol (Pluronic®, BASF), polyethylene oxide, derivatives of methacrylates, polyvinyl alcohol and/polyethylene glycol, and mixtures of the polymers mentioned.

The matrix material particularly preferably used is polyvinyl pyrrolidone, preferably with a weight-average molecular weight of 10,000 to 60,000 g/mol, especially 12,000 to 40,000 g/mol, copolymer of vinyl pyrrolidone and vinyl acetate, especially with a weight-average molecular weight of 40,000 to 70,000 g/mol and/or polyethylene glycol, especially with a weight-average molecular weight of 2,000 to 10,000 g/mol.

Further examples of possible hydrophilic polymers for the matrix material comprise: polysaccharides, such as hydroxypropyl methyl cellulose (HPMC), carboxymethyl cellulose (CMC, especially sodium and calcium salts), ethyl cellulose, methyl cellulose, hydroxyethyl cellulose, ethyl hydroxyethyl cellulose, hydroxypropyl cellulose (HPC); microcrystalline cellulose, and mixtures of the polymers mentioned; or mixtures of the polymers mentioned with polymers listed above.

If HPMC is used, it is preferably HPMC with a weight-average molecular weight of 20,000 to 90,000 g/mol and/or preferably a proportion of methyl groups of 10 to 35% and a proportion of hydroxy groups of 1 to 35%. In addition, microcrystalline cellulose can be used, especially one with a specific surface area of 0.7-1.4 m²/g. The specific surface area is determined by means of the gas adsorption method according to Brunauer, Emmet and Teller.

Furthermore, the matrix material also includes solid, non-polymeric compounds which preferably contain polar side groups. Examples of these are sugar alcohols or disaccharides. Examples of suitable sugar alcohols and/or disaccharides are mannitol, sorbitol, xylitol, isomalt, glucose, fructose, maltose and mixtures thereof. The term “sugar alcohols” in this context also includes monosaccharides. In particular, isomalt and sorbitol can be used as the matrix material.

In addition, the matrix material also includes substances which behave like polymers. Examples of these are fats and waxes. It is, for example, possible to use waxes, such as cetyl palmitate, carnauba wax or bees' wax, as the matrix material. It is likewise possible to use fats, such as glycerol fatty acid esters (e.g. glycerol palmitate, glycerol behenate, glycerol laurate, glycerol stearate), PEG glycerol fatty acid esters or vegetable oils or hydrogenated vegetable oils. Further examples of matrix materials are glycerol, stearyl alcohol, salts of fatty acids (e.g. aluminium monostearate).

Apart from that, natural gum can be used as the matrix material, e.g. gum traganth, alginates, gum arabic, gum guar.

The matrix material may contain one or more of the above-mentioned substances.

In a preferred embodiment, the intermediate of the invention contains fingolimod and matrix material, the weight ratio of fingolimod to matrix material being 2:1 to 1:200, more preferably 1:2.5 to 1:150, even more preferably 1:5 to 1:120, especially 1:5 to 1:100. Weight ratios of 1:10 or 1:15 to 1:20 are particularly preferable, especially ratios of 1:10, 1:15 and 1:20 of fingolimod to matrix material.

It is preferable that the type and quantity of the matrix material should be selected such that the resulting intermediate has a glass transition temperature (Tg) of more than 20° C., preferably >25° C.

It is preferable that type and quantity of the polymer should be selected such that the resulting intermediate is storage-stable. “Storage-stable” means that in the intermediate of the invention, after storage for 3 years at 25° C. and 50% relative humidity, the proportion of crystalline fingolimod—based on the total amount of fingolimod—is no more than 60% by weight, preferably no more than 30% by weight, more preferably no more than 15% by weight, in particular no more than 5% by weight.

In a preferred embodiment, the intermediate only contains fingolimod and one or more of the substances listed above as matrix material.

In an alternative preferred embodiment, in addition to fingolimod and matrix material, the intermediates of the invention also contain a crystallisation inhibifor based on an inorganic salt, an organic acid or a high-molecular-weight polymer with an average molecular weight of more than 500,000 g/mol.

These polymers which are suitable as crystallisation inhibitors are also referred to in the context of this invention as “high-viscosity polymers”. Their weightaverage molecular weight is usually less than 5,000,000 g/mol. A preferred high-viscosity polymer is povidone.

The crystallisation inhibitor is preferably ammonium chloride, citric acid, or Povidone K 90 (in accordance with Ph. Eur. 6.0).

The crystallisation inhibitor can generally be used in an amount of 1 to 30% by weight, preferably 2 to 25% by weight, more preferably 5 to 20% by weight, based on the total weight of the intermediate.

The intermediates of the invention are obtainable by a variety of preparation methods. Depending on the preparation method, the intermediates are obtained in different particle sizes. Normally, the intermediates of the invention are present in particulate form and have an average particle diameter (D50) of 1 to 750 μm, depending on the preparation method.

The expression “average particle diameter” relates in the context of this invention to the D50 value of the volume-average particle diameter determined by means of laser diffractometry. In particular, a Malvern Instruments Mastersizer 2000 was used to determine the diameter (wet measurement with ultrasound for 60 sec., 2,000 rpm, the evaluation using the Fraunhofer method, and preferably using a dispersant in which the substance to be measured does not dissolve at 20° C.). The average particle diameter, which is also referred to as the D50 value of the integral volume distribution, is defined in the context of this invention as the particle diameter at which 50% by volume of the particles have a smaller diameter than the diameter which corresponds to the D50 value. Similarly, 50% by volume of the particles then have a larger diameter than the D50 value.

Another subject matter of the invention is a method of preparing the intermediate of the invention. In the following, two preferred embodiments of such a method will be explained.

In a first preferred embodiment, the invention relates to a spray-drying or freeze-drying method of preparing the intermediate of the invention, comprising the steps of
(a1) dissolving fingolimod and the matrix material in a solvent or mixture of solvents, and
(b1) spray-drying or freeze-drying the solution from step (a1).

In step (a1), fingolimod and the matrix material described above, is dissolved, preferably completely dissolved, in a solvent or mixture of solvents. Crystalline or amorphous fingolimod may be used. Preferably, crystalline fingolimod is used.

Suitable solvents are, for example, water, alcohol (e.g. methanol, ethanol, isopropanol), dimethyl sulphoxide (DMSO), acetone, butanol, ethyl acetate, heptane, pentanol or mixtures thereof. Preferably, an ethanol/water mixture is used, or water mixed with a different solvent, such as one of the above-mentioned solvents which is miscible with water.

Suitable matrix materials in this embodiment are especially polyvinyl pyrrolidone and copolymers thereof (preferably with a weight-average molecular weight of 20,000 to 70,000 g/mol) and sugar alcohols such as isomalt and sorbitol.

If the intermediate to be prepared is additionally intended to contain a crystallisation inhibitor based on an inorganic salt or an organic acid, or a highly viscous polymer, this can be added in step (a1). Reference is made to the above observations with regard to the type and amount of the crystallisation inhibitor.

In the subsequent step (b1), the solution from step (a1) is spray-dried or freezedried. The spray-drying is usually carried out in a spray tower. As an example, a Büchi B-191 is suitable (Büchi Labortechnik GmbH, Germany). Preferably, an inlet temperature of 100° C. to 150° C. is chosen. The amount of air is, for example, 500 to 700 litres/hour, and the aspirator preferably runs at 80 to 100%. Spray-drying has the advantage of a continuous method, which enhances the reproducibility and hence also the homogeneity and uniformity of content of active agent. Freeze-drying is usually carried out in a freezer-dryer, for example a VirTis®Benchtop K Freeze Dryer. Generally, the freeze-drying process comprises two stages. Stage 1: Freezing the solution and reducing the pressure, preferably below the triple point of the solution. Stage 2: Raising the temperature, preferably to the sublimation curve, in order to allow latent heat of sublimation. After the sublimation is complete, the freeze-dried (lyophilised) substrate is warmed to room temperature.

The process conditions in this first embodiment are preferably selected such that the resulting intermediate particles have a volume-average particle diameter (D50) of 1 to 250 μm, more preferably 2 to 150 μm, especially 3 to 100 μm.

In a second preferred embodiment, the invention relates to a melt extrusion process, i.e. a method of preparing the intermediate of the invention, comprising the steps of

(a2) mixing fingolimod and matrix material, and
(b2) extruding the mixture.

Of the preparation methods described, the second embodiment is particularly preferable. It, too, permits a continuous process, which improves the reproducibility of the method as a whole, and hence also the uniformity of content of active agent in the intermediate and products prepared from it.

In step (a2), fingolimod is mixed with the matrix material, preferably in a mixer. In this embodiment of the method of the invention, a matrix material in polymeric form is used. Crystalline or amorphous fingolimod may be used. Preferably, crystalline fingolimod is used.

Suitable polymeric matrix materials in this embodiment are especially polyvinyl pyrrolidone and vinyl pyrrolidone/vinyl acetate copolymers, and also polyvinyl alcohols, methacrylates and HPMC. The weight-average molecular weight of the polymers used is usually 20,000 to 90,000 g/mol. Alternatively, a sugar alcohol, especially isomalt, can also be used.

If the intermediate to be prepared is additionally intended to contain a crystallisation inhibitor based on an inorganic salt or an organic acid, or a highly viscous polymer, this can likewise be added in step (a2). Reference is made to the above observations with regard to the type and amount of the crystallisation inhibitor.

The mixture from step (a2) is conventionally processed in the extruder into a homogeneous melt. In step (b2), the mixture is extruded.

Conventional melt extruders can be used as the extruders. The screw profile of the extruder preferably contains kneading units. The shear forces created in this way contribute to melting the mixture and thus to dissolving the active agent in the matrix material. By way of example, a Leistritz Micro 18 is used.

The extrusion temperature depends on the nature of the matrix material. It usually lies between 50 and 250° C., preferably between 60 and 150° C., more preferably between 80 and 120° C. The extrusion is preferably carried out at an outlet pressure of 10 bar to 100 bar, more preferably at 20 to 80 bar.

The cooled melt is usually comminuted by a rasp screen (e.g. Comil® U5) and in this way reduced to a uniform particle size.

The process conditions in this second embodiment are preferably selected such that the resulting intermediate particles have a volume-average particle diameter (D50) of 150 to 1,000 μm, more preferably a D50 of 250 to 800 μm.

Instead of granulating the extruded material, “direct injection moulding” may also be performed. In this case, the method of the invention includes the step of
(c2) injection moulding the extruded material into moulds for pharmaceutical dosage forms.

In a further embodiment, the intermediate is produced by means of lyophilisation.

Examples are moulds for tablets.

The intermediate of the invention (i.e. the molecularly disperse fingolimod of the invention) is usually employed to prepare a pharmaceutical formulation.

The subject matter of the invention is therefore a pharmaceutical formulation containing intermediate of the invention and pharmaceutical excipients, or fingolimod in the form of a solid solution in a matrix material.

The pharmaceutical formulation may be present, for example, in the form of sachets, capsules or tablets. Tablets are preferable. It is also preferable that the pharmaceutical formulations are intended for oral administration, especially for peroral administration (for swallowing).

The pharmaceutical excipients are excipients with which the person skilled in the art is familiar, such as those which are described in the European Pharmacopoeia.

Examples of pharmaceutical excipients used are disintegrants, anti-stick agents, emulsifiers, pseudo-emulsifiers, fillers, additives to improve the powder flowability, glidants, wetting agents, gelling agents and/or lubricants. Where appropriate, further excipients can also be used.

The ratio of active agent to excipients is preferably selected such that the resulting pharmaceutical formulations contain 0.1 to 4% by weight, more preferably 0.12 to 2.5% by weight, especially 0.12 to 1.75% by weight, more preferably 0.15 to 1.0% by weight, especially 0.25 to 0.4% by weight fingolimod, and accordingly 99.9 to 96% by weight excipients, more preferably 99.88 to 97.5% by weight, especially 99.88 to 98.25% by weight, more preferably 99.85 to 99.0% by weight, especially 99.75 to 99.6% by weight excipients.

In these ratios specified, the amount of matrix former optionally used to prepare the intermediate of the invention is counted as an excipient. This means that the amount of active agent refers to the amount of fingolimod contained in the formulation.

The intermediate preferably accounts for 1.25 to 20% by weight of the total weight of the formulation, more preferably 2.0 to 15.0% by weight, even more preferably 2.5 to 10% by weight and especially 3.0 to 8% by weight. This applies to all the embodiments, irrespective of the nature of the pharmaceutical excipients apart from the intermediate.

It has been shown that the intermediates of the invention are suitable for serving both as a basis for a dosage form with immediate release (or “IR” for short) and also with modified release (or “MR” for short).

In a preferred embodiment for an IR formulation, a relatively large amount of disintegrant is used. In that preferred embodiment, the pharmaceutical formulation of the invention therefore contains
(i) 1.25 to 20% by weight, more preferably 2.5 to 10% by weight, especially 3 to 8% by weight intermediate and
(ii) 5 to 30% by weight, more preferably 6 to 25% by weight, especially 7 to 20% by weight disintegrants, based on the total weight of the formulation.

“Disintegrants” is the term generally used for substances which accelerate the disintegration of a dosage form, especially a tablet, after it is placed in water. Suitable disintegrants are, for example, organic disintegrants such as carrageenan, croscarmellose (including croscarmellose sodium), sodium carboxymethyl cellulose, sodium carboxymethyl starch and crospovidone. Alkaline disintegrants are likewise used. The term “alkaline disintegrants” means disintegrants which, when dissolved in water, produce a pH level of more than 7.0.

It is also possible to use inorganic alkaline disintegrants, especially salts of alkali and alkaline earth metals. Preferred examples here are sodium, potassium, magnesium and calcium. As anions, carbonate, hydrogen carbonate, phosphate, hydrogen phosphate and dihydrogen phosphate are preferred. Examples are sodium hydrogen carbonate, sodium hydrogen phosphate, calcium hydrogen carbonate and the like.

Sodium carboxymethyl starch or sodium carboxymethyl cellulose, particularly preferably sodium carboxymethyl starch, are particularly preferably used as disintegrants, especially in the above-mentioned amounts.

In a preferred embodiment for an MR formulation, a relatively small amount of disintegrant is used. In that preferred embodiment, the pharmaceutical formulation of the invention therefore contains
(i) 1.25 to 20% by weight, more preferably 2.5 to 10% by weight, especially 3 to 8% by weight intermediate and
(ii) 0 to 10% by weight, more preferably 0.1 to less than 5% by weight, especially 1 to 4% by weight disintegrants, based on the total weight of the formulation.

In the case of the MR formulation, sodium carboxymethyl starch or sodium carboxymethyl cellulose is preferred as the disintegrant.

In addition, the conventional retardation techniques can be used for the MR formulation.

In a preferred embodiment, the formulation of the invention contains 2 to 8% by weight, more preferably 3 to 7% by weight, especially 4 to 6% by weight anti-stick agent, based on the total weight of the formulation. This embodiment is used especially for the production of tablets.

“Anti-stick agents” is usually understood to mean substances which reduce agglomeration in the core bed. Examples are talcum, silica gel, polyethylene glycol (preferably with 2,000 to 10,000 g/mol weight-average molecular weight) and/or glycerol monostearate.

Furthermore, the pharmaceutical formulation (both for IR and for MR) preferably contains one or more of the above-mentioned excipients. These will be explained in more detail below.

The formulation of the invention preferably contains fillers. “Fillers” generally means substances which serve to form the body of the tablet in the case of tablets with small amounts of active agent (e.g. less than 70% by weight). This means that fillers “dilute” the active agents in order to produce an adequate tablet-compression mixture. The normal purpose of fillers, therefore, is to obtain a suitable tablet size.

Examples of preferred fillers are lactose, lactose derivatives, starch, starch derivatives, microcrystalline cellulose, treated starch, talcum, calcium phosphate, sucrose, calcium carbonate, magnesium carbonate, magnesium oxide, maltodextrip, calcium sulphate, dextrates, dextrin, dextrose, hydrogenated vegetable oil, kaolin, sodium chloride, and/or potassium chloride. Prosolv® (Rettenmaier & Söhne, Germany) can likewise be used.

Fillers are normally used in an amount of 1 to 99% by weight, more preferably 30 to 95% by weight, based on the total weight of the formulation. In addition, it is, for example, possible for at least 40% by weight or at least 50% by weight filler to be used.

One example of an additive to improve the powder flowability is disperse or colloidal silica, e.g. known under the trade name Aerosil®.

Additives to improve the powder flowability are generally used in an amount of 0.1 to 3% by weight, based on the total weight of the formulation.

In addition, lubricants may be used. Lubricants are generally used in order to reduce sliding friction. In particular the intention is to reduce the sliding friction found during tablet pressing between the punch moving up and down in the die and the die wall, on the one hand, and between the edge of the tablet and the die wall, on the other hand. Suitable lubricants are, for example, stearic acid, adipic acid, sodium stearyl fumarate (Pruv®) and/or magnesium stearate.

Lubricants are generally used in an amount of 0.1 to 3% by weight, based on the total weight of the formulation.

The examples provided here for matrix material and the other excipients are optional, i.e. they may be used in the intermediates and formulations of the invention, but embodiments are of course also encompassed which are free of one or more of the substances or combinations of substances mentioned as examples.

The pharmaceutical formulation of the invention is preferably compressed into tablets.

The intermediates of the invention can therefore be compressed into tablets by means of direct compression or are subjected to dry granulation before being compressed into tablets. Intermediates with a bulk density of less than 0.5 g/ml are preferably processed by dry granulation.

Direct compression is especially preferred if the intermediate is prepared by means of melt extrusion (process steps (a2) and (b2)).

Dry granulation is especially preferred if the intermediate is prepared by means of spray-drying (process steps (a1) and (b1)).

In a further aspect, the present invention therefore relates to a dry granulation process, i.e. a method of preparing granules, comprising the steps of
(I) preparing the intermediate of the invention and one or more pharmaceutical excipients (especially those described above);
(II) compacting it into flakes; and
(III) granulating or comminuting the flakes into granules.

In step (I), fingolimod in the form of the solid solution (i.e. in the form of the intermediate of the invention) and excipients are preferably mixed. The mixing can be performed in conventional mixers. Alternatively, it is possible that the fingolimod of the invention is initially only mixed with part of the excipients (e.g. 50 to 95%) before compacting (II), and that the remaining part of the excipients is added after the granulation step (III). In the case of multiple compacting, the excipients should preferably be mixed in before the first compacting step, between multiple compacting steps or after the last granulation step.

In step (II) of the method of the invention, the mixture from step (I) is compacted into flakes. It is preferable here that it should be dry compacting, i.e. the compacting is preferably performed in the absence of solvents, especially in the absence of organic solvents.

The compacting conditions are, for example, selected such that the intermediate of the invention is present in the form of compacted material (flakes), the density of the intermediate (or the flakes) being 0.8 to 1.3 g/cm³, preferably 0.9 to 1.20 g/cm³, especially 1.01 to 1.15 g/cm³.

The term “density” here preferably relates to the “pure density” (i.e. not to the bulk density or tapped density). The pure density can be determined with a gas pycnometer. The gas pycnometer is preferably a helium pycnometer; in particular, the AccuPyc 1340 helium pycnometer from the manufacturer Micromeritics, Germany, is used.

The compacting is preferably carried out in a roll granulator.

The rolling force is preferably 5 to 70 kN/cm, preferably 10 to 60 kN/cm, more preferably 15 to 50 kN/cm.

The gap width of the roll granulator is, for example, 0.8 to 5 mm, preferably 1 to 4 mm, more preferably 1.5 to 3 mm, especially 1.8 to 2.8 mm.

The compacting apparatus used preferably has a cooling means. In particular, the cooling is preferably such that the temperature of the compacted material does not exceed 50° C., especially 40° C.

In step (III) of the method, the flakes are granulated, or comminuted into granules. The granulation can be performed with methods known in the prior art.

In a preferred embodiment, the granulation conditions are selected such that the resulting particles (granules) have a volume-average particle size (D50) value) of 50 to 800 μm, more preferably 100 to 750 μm, even more preferably 150 to 500 μm, especially 200 to 450 μm.

In a preferred embodiment, the granulation is performed in a screen mill. In this case, the mesh width of the screen insert is usually 0.1 to 5 mm, preferably 0.5 to 3 mm, more preferably 0.75 to 2 mm, especially 0.8 to 1.8 mm.

In a preferred embodiment, the method is adapted such that multiple compacting occurs, with the granules resulting from step (III) being returned once or more times to the compacting (II). The granules from step (III) are preferably returned 1 to 5 times, especially 2 to 3 times.

The granules resulting from step (IV) can be further used or processed into pharmaceutical dosage forms. For this purpose, the granules are filled into sachets or capsules. The granules resulting from step (III) are, however, preferably compressed into tablets (=step 1V).

This means that a further subject matter of the invention is a method of preparing a tablet, comprising the process of preparing granules, and further comprising the following step:
(IV) compressing the granules, and optionally one or more additional pharmaceutical excipients, into a tablet.

In step (IV) of the method, the granules obtained in step (III) are compressed into tablets, i.e. the step involves compression into tablets. Compression can be performed with tableting machines known in the prior art.

In step (IV) of the method, pharmaceutical excipients may optionally be added to the granules from step (III).

The amounts of excipients which may be added in step (IV) usually depend on the type of tablet to be produced and the amount of excipients which were already added in steps (I) or (II). This preferably involves the addition or one or more lubricants, such as those already described above.

In the case of direct compression, only steps (I) and (IV) of the method described above are performed. This means that a further subject matter of the invention is a method of preparing a tablet, comprising the following steps:
(I) preparing, and optionally mixing, the intermediate of the invention and one or more pharmaceutical excipients (especially those described above);
(IV) compressing the intermediate and the one or more pharmaceutical excipients into a tablet.

The method preferably does not involve any further steps between these two steps.

The tableting conditions are preferably selected such that the resulting tablets have a tablet height to weight ratio of 0.005 to 0.03 mm/mg, more preferably 0.006 to 0.02 mm/mg, particularly preferably 0.007 to 0.015 mm/mg.

In accordance with the invention, the resulting tablets preferably have a mass of 100 to 550 mg, such as 110 to 350 mg, 120 to 250 mg, 125 to 240 mg or particularly preferably 130 to 220 mg.

In addition, the resulting tablets preferably have a hardness of 50 to 200 N, particularly preferably 80 to 150 N. The hardness is determined in accordance with Ph. Eur. 6.0, section 2.9.8.

In addition, the resulting tablets preferably have a friability of less than 5%, particularly preferably less than 3%, especially less than 2%. The friability is determined in accordance with Ph. Eur. 6.0, section 2.9.7.

Finally, the tablets of the invention usually exhibit a content uniformity of fingolimod, determined in accordance with Ph. Eur. 2.9.6, which is characterised in that each of ten dosage form units has a content of fingolimod which lies between 90 and 110%, preferably 95 to 105%, especially 98 to 102% of the average content of those ten dosage form units.

In particularly preferred embodiments, fingolimod is contained in the formulation in amounts of 0.5 mg, 0.75 mg, 1 mg, 1.25 mg, 1.5 mg, 1.75 mg, 2 mg or 2.5 mg.

In the case of an IR formulation, the release profile of the tablets of the invention according to the USP method (USP basket apparatus, 500 ml test medium; 0.1 N HCl and 0.2% sodium dodecyl sulfate, 37° C. and 100 rpm) after 10 minutes usually indicates a content released of at least 30%, preferably at least 60%, especially at least 98%.

In the case of an MR formulation, the release profile of the tablets of the invention according to the USP method (USP basket apparatus, 500 ml test medium; 0.1 N HCl and 0.2% sodium dodecyl sulfate, 37° C. and 100 rpm) after 10 minutes indicates, for example, a content released of no more than 98%, preferably no more than 90%, further preferably no more than 75%, more preferably no more than 50% and particularly preferably no more than 30%.

The above details regarding hardness, friability, content uniformity and release profile preferably relate here to the non-film-coated tablet for an IR formulation. For a modified-release tablet, the release profile relates to the total formulation.

The tablets produced by the method of the invention are preferably tablets for oral administration and specifically ones which can be swallowed unchewed (non-film-coated or preferably film-coated).

In the case of tablets which are swallowed unchewed, it is preferable that they be coated with a film layer. For this purpose, the methods of film-coating tablets which are standard in the prior art may be employed. The above-mentioned ratios of active agent to excipient, however, relate to the non-film-coated tablet.

For film-coating, macromolecular substances are preferably used, such as modified celluloses, polymethacrylates, polyvinyl pyrrolidone, polyvinyl acetate phthalate, zein and/or shellack or natural gum, such as carrageenan.

The thickness of the coating is preferably 1 to 100 μm.

Because of the advantageous properties of the intermediate, the present intermediates, or the formulations containing them, are particularly suitable for comedication. This means that they are particularly suitable for administration with a pharmaceutical formulation containing an active agent which is different from fingolimod and can likewise be taken orally. In this context, they are partitularly advantageous when administered together with a pharmaceutical formulation with an active agent which is different from fingolimod and which is suitable for modifying the pH at the site of absorption of fingolimod. This means that the intermediates and formulations of the invention are suitable, for example, for administration together with proton pump inhibitors, such as omeprazol, esomeprazol, lansoprazol, pantoprazol or rabeprazol. At the same time, they are also suitable for use together with psychotropic drugs, such as antidepressants. An antidepressant for administration together with the intermediates or formulations of the invention may, for example, be selected from the group of serotonin re-uptake inhibitors (SSRI), tricyclic antidepressants, monoamino-oxidase inhibitors and benzodiazepines. The administration of two formulations together includes simultaneous administration, but also administration spaced out over a time of up to three hours. If one of the formulations is one with modified release (MR), administration together can also cover a longer period. The period during which the formulation administered later in time can still be administered advantageously comprises at least the time required for the release, according to the USP method, of 90% of the active agent administered as the first formulation, plus three, preferably 1.5 hours.

The invention therefore relates, according to a further aspect, to a pharmaceutical formulation as described above, for administration to patients taking one or more proton pump inhibitors and/or an antidepressant or a number of antidepressants, especially those who take such drugs regularly, i.e. over a period of more than two days.

The invention will now be illustrated with reference to the following examples.

### EXAMPLES

### Example 1a

### Preparation of an Intermediate by Melt Extrusion and Subsequent Compression into Tablets

Fingolimod was mixed with Pluronic® in a ratio of 1:20 and melted in the melt extruder at temperatures of less than 120° C. and extruded in a temperature cascade. A die plate with a hole diameter of 1 mm was used. The Leistritz® micro 18 twin-screw extruder was equipped with various screw elements. A kneading unit ensured the necessary thorough mixing and dissolution of the active agent in the Pluronic®.

After cooling and screening through a screen with a mesh width of 0.71 mm, Avicel®, sodium carboxymethyl starch and colloidal silica were added to the intermediate (extruded material). After that, the mixture was mixed for 15 minutes in a free-fall mixer (Turbula® T10B). Magnesium stearate was added through a screen with a mesh width of 0.5 mm and mixed for a further 3 minutes. The resulting mixture was then compressed into tablets (Riva Piccolo®). These tablets have the following composition:

| | |
|---|---|
| fingolimod | 0.5 mg |
| Pluronic ® 68 | 10 mg |
| MCC | 120 mg |
| sodium carboxymethyl starch | 15 mg |
| colloidal silica | 4 mg |
| magnesium stearate | 2 mg |

### Example 1b

Tablets were produced according to Example 1a, except that the excipients sodium carboxymethyl starch, colloidal silica and magnesium stearate were substituted by sodium starch glycolate, silica and sodium stearyl fumarate. Thus, the tablets have the following composition:

| | |
|---|---|
| fingolimod | 0.5 mg |
| Pluronic ® 68 | 10 mg |
| MCC | 120 mg |
| sodium starch glycolate | 15 mg |
| silica | 4 mg |
| sodium stearyl fumarate | 2 mg |

### Example 1c

Tablets were produced according to Example 1b, except that MCC was substituted by Lactose (Tablettose® 100). Thus, the tablets have the following composition:

| | |
|---|---|
| fingolimod | 0.5 mg |
| Pluronic ® 68 | 10 mg |
| Lactose (Tablettose ® 100) | 120 mg |
| sodium starch glycolate | 15 mg |
| silica | 4 mg |
| sodium stearyl fumarate | 2 mg |

### Example 2

### Preparation of an Intermediate by Spray-Drying and Subsequent Compression into Modified-Release Tablets

Fingolimod was dissolved in water/methanol together with Eudragit RS/RL 70/30 (in a ratio of 1:10). That solution was spray-dried in a Büchi spray tower using the following parameters:

spray pressure: 3 to 4 bar
nozzle: 1.4 mm
aspirator: 90%

After screening through a screen with a mesh width of 0.71 mm, microcrystalline cellulose, corn starch, sodium carboxymethyl starch and colloidal silica were added to the intermediate. After that, the mixture was mixed for 15 minutes in a free-fall mixer (Turbula® T10B). Magnesium stearate was added through a screen with a mesh width of 0.5 mm and mixed for a further 3 minutes. The resulting mixture was then compressed into tablets (Riva Piccolo®).

These tablets have the following composition:

| | |
|---|---|
| fingolimod | 0.5 mg |
| Kollidon ® VA 64 | 5 mg |
| microcrystalline cellulose | 80 mg |
| corn starch | 40 mg |
| sodium carboxymethyl starch | 3 mg |
| colloidal silica | 2 mg |
| magnesium stearate | 1 mg |

1. An intermediate containing fingolimod and matrix material, wherein the fingolimod is present in the matrix material in the form of a solid solution.

2. The intermediate as claimed in claim 1 wherein the weight ratio of fingolimod to matrix material is 1:1 to 1:200.

3. The intermediate of claim 1, characterised in that the matrix material is a polymer, preferably a polymer with a glass transition temperature (Tg) higher than 15° C.

4. The intermediate of claim 1, characterised in that the matrix material is at least a hydrophilic polymer selected from the group consisting of polyvinyl pyrrolidone, polyvinyl acetate (PVAC), polyvinyl alcohol (PVA), polymers of acrylic acid and their salts, polyacrylamide, polymethacrylates, vinyl pyrrolidone/vinyl acetate copolymers, polyalkylene glycols, polypropylene glycol, polyethylene glycol, co-block polymers of polyethylene glycol, coblock polymers of polyethylene glycol and polypropylene glycol, and polyethylene oxide.

5. The intermediate of claim 1, characterised in that the glass transition temperature (Tg) of the intermediate is more than 20° C.

6. The intermediate of claim 1, characterised in that it additionally comprises a crystallisation inhibitor based on an inorganic salt, an organic acid, a highviscosity polymer or mixtures thereof.

7. The intermediate as claimed in claim 6, wherein the crystallisation inhibitor is citric acid, ammonium chloride, povidone with a weight-average molecular weight of at least 700,000 g/mol or mixtures thereof.

8. A method of preparing an intermediate of claim 1, comprising the steps of
(a1) dissolving fingolimod and the matrix material in a solvent or mixture of solvents, and
(b1) spray-drying or freeze-drying the solution from step (a1).

9. A method of preparing an intermediate of claim 1, comprising the steps of
(a2) mixing fingolimod and matrix material, and
(b2) extruding the mixture.

10. (canceled)

11. A pharmaceutical formulation comprising fingolimod in the form of an intermediate as claimed in claim 1, and optionally at least one further pharmaceutical excipient.

12. The pharmaceutical formulation as claimed in claim 11, which is present as a capsule or tablet for oral administration.

13. The pharmaceutical formulation as claimed in claim 11, comprising
(i) 1.25 to 20% by weight intermediate and
(ii) 0.1 to 10% by weight disintegrants, based on the total weight of the formulation.

14. The pharmaceutical formulation as claimed in claim 13, characterised in that the disintegrants are sodium carboxymethyl starch or sodium carboxymethyl cellulose.

15. The pharmaceutical formulation of claim 11, containing 2 to 8% by weight anti-stick agents, based on the total weight of the formulation.

16. The pharmaceutical formulation of claim 11, for administration with a pharmaceutical formulation containing an active agent different from fingolimod.

17. A method of identifying a pharmaceutical excipient which is suitable as a matrix material for fingolimod in the form of a solid solution, comprising the steps of:
a) preparing fingolimod, a pharmaceutical excipient which is present in a solid aggregate state at 25° C., and a 1:1 mixture of fingolimod and excipient;
b) twice heating up the solid excipient by means of DSC and identifying the glass transition temperature of the excipient (Tg_{Excip});
c) twice heating up the active agent fingolimod by means of DSC and identifying the glass transition temperature of the active agent (Tg_{Fingo});
d) twice heating up a 1:1 mixture of fingolimod and excipient by means of DSC and identifying the glass transition temperature of the mixture (Tg_{Mix}), and
e) selecting the excipient as “suitable” provided that Tg_{Mix} is between Tg_{Excip} and Tg_{Fingo}.

18. An intermediate of molecularly disperse fingolimod and a pharmaceutical excipient as the matrix material, the excipient being identified in accordance with a method as claimed in claim 17, and wherein the weight ratio of fingolimod to matrix material is preferably 1:1 to 1:200.

19. A method of preparing granules, comprising the steps of
(I) preparing the intermediate of claim 1 and one or more pharmaceutical excipients;
(II) compacting the intermediate with the one or more excipients into flakes; and
(III) comminuting the flakes into granules.

20. A method of preparing a tablet, comprising the method of preparing granules as claimed in claim 19, and further comprising the following step:
(IV) compressing the granules, and optionally one or more additional pharmaceutical excipients, into a tablet.

21. A method of preparing a tablet, comprising the following steps:
(I) preparing, and optionally mixing, the intermediate of claim 1 and one or more pharmaceutical excipients;
(IV) compressing the intermediate and the one or more pharmaceutical excipients into a tablet.
